(19) United States
(12) Patent Application Publication
(10) Pub. No.: US 2024/0207614 A1
(43) Pub. Date: Jun. 27, 2024

(54) IMPLANTATION OF NEUROSTIMULATORS

(71) Applicant: BLUEWIND MEDICAL LTD., Herzlia (IL)

(72) Inventors: Shahar BARUCH, Kfar Harif (IL); Karin AHARONSON-RAZ, Sandy, UT (US); Gur ORON, Tel Aviv (IL); Roy KATZ, Lincoln, MA (US)

(73) Assignee: BLUEWIND MEDICAL LTD., Herzlia (IL)

(21) Appl. No.: 18/391,128

(22) Filed: Dec. 20, 2023

Related U.S. Application Data
(60) Provisional application No. 63/434,699, filed on Dec. 22, 2022.

(51) Int. Cl. A61N 1/36

(52) U.S. Cl. CPC A61N 1/36132

(57) ABSTRACT

A method is provided including placing a nerve stimulator implant at an implant location in a patient's body in a vicinity of a target nerve. The nerve stimulator implant is activated to apply electrical stimulation to the target nerve. The following indications are received from the patient: (a) a remote-paresthesia indication of a presence or an absence of remote-site paresthesia caused by the electrical stimulation at a predetermined remote-paresthesia site of the body that is remote from the implant location, and (b) an adverse-effect indication of a presence or an absence of an adverse sensation caused by the electrical stimulation at an adverse-sensation site of the body that is not innervated by the portion of the target nerve. Depending on the indications, the nerve stimulator implant is adjusted, the implant location is adjusted, or the implantation of the nerve stimulator implant is completed without adjustment. Other embodiments are also described.

### CROSS-REFERENCE TO RELATED APPLICATIONS

The present application claims the benefit of US Provisional Application 63/434,699, filed Dec. 22, 2022, which is assigned to the assignee of the present application and incorporated herein by reference.

### FIELD OF THE APPLICATION

The present invention relates generally to implantation and programming techniques for neurostimulators.

### BACKGROUND OF THE APPLICATION

Neurological disorders affect the nerves, muscles or the brain. Many neurological disorders reduce or eliminate voluntary recruitment of muscles, which may result in loss of ability to perform motor tasks or to maintain systems that depend on muscle activity for their function. Many neurological disorders cause pain.

Neurostimulation is a clinical tool used to treat various neurological disorders. This technique involves modulation of the nervous system by electrically activating fibers in the body. Percutaneous implants exist for providing neurostimulation.

### SUMMARY OF THE APPLICATION

Some embodiments of the present invention provide a method for intraprocedural adjustment of a nerve stimulator implant during implantation in a body of a patient. During the implantation, a healthcare professional uses patient-reported paresthesia at a site remote from the implantation site as an indicator that the implant is properly placed, while selecting against sites that also yield patient-reported sensation at the stimulation site or other non-target sites. Thus, for example, if the healthcare professional observes that a target nerve (e.g., the tibial nerve) is indeed being stimulated, but the patient reports sensation at the stimulation site, the healthcare professional adjusts the implant location to find an implant location from which the target nerve is stimulated while the patient does not report stimulation-site sensation.

Some embodiments of the present invention provide a method for performing configuration (i.e., programming or reprogramming) of an implanted nerve stimulator implant. After the implantation, the healthcare receives patient-reported feedback of where on a plantar surface of a foot the patient feels paresthesia caused by the electrical stimulation paresthesia. Based on the feedback, the healthcare professional ascertains at which dermatome (s) of the plantar surface the patient felt the paresthesia, and adjusts one or more stimulation parameters in an attempt to maximize coverage of the dermatomes, optionally including a preference for particular dermatome (s) if it is not possible to stimulate as many dermatomes as desired.

There is therefore provided, in accordance with an application of the present invention, a method for performing an implantation of a nerve stimulator implant in a body of a patient, the method including:
(i) placing the nerve stimulator: implant at an implant location in the body in a vicinity of a target nerve;
(ii) activating the nerve stimulator implant to apply electrical stimulation to a portion of the target nerve;
(iii) receiving from the patient:
   (a) a remote-paresthesia indication of at least a presence or an absence of remote-site paresthesia caused by the electrical a stimulation at predetermined remote-paresthesia site of the body that is (1) innervated by the portion of the target nerve, (2) remote from the implant location, and (3) not a therapeutic target of stimulation by the nerve stimulator implant, and
   (b) an adverse-effect indication of at least a presence or an absence of an adverse sensation caused by the electrical stimulation at an adverse-sensation site of the body that is not innervated by the portion of the target nerve; and
(iv) in response to:
   (a) the remote-paresthesia indication being of the absence of the remote-site paresthesia, adjusting the nerve stimulator implant and thereafter repeating steps (iii) and (iv),
   (b) the adverse-effect indication being of the presence of the adverse sensation, adjusting the implant location of the nerve stimulator implant and thereafter repeating steps (iii) and (iv), and
   (c) both (1) the remote-paresthesia indication being indicative of the presence of the remote-site paresthesia and (2) the adverse-effect indication being indicative of the absence of the adverse sensation, completing the implantation of the nerve stimulator implant at the implant location without repeating steps (iii) or (iv).

For some applications, adjusting the nerve stimulator implant at step (iv) (a) includes adjusting the implant location of the nerve stimulator implant.

For some applications, adjusting the nerve stimulator implant at step (iv) (a) includes configuring one or more parameters of the electrical stimulation to increase an implantation-procedure strength the electrical stimulation applied by the nerve stimulator implant.

For some applications, adjusting the nerve stimulator implant at step (iv) (a) includes:
if the nerve stimulator implant is programmed with one or more parameters of the electrical stimulation that produce an implantation-procedure strength of the electrical stimulation that is less than a predetermined maximum implantation-procedure strength of the electrical stimulation, configuring the one or more parameters to increase the implantation-procedure strength the electrical stimulation, and
if the nerve stimulator implant is programmed with the one or more parameters that produce the predetermined maximum implantation-procedure strength of the electrical stimulation, adjusting the implant location of the nerve stimulator implant.

For some applications, adjusting the nerve stimulator implant at step (iv) (a) includes:
if a programmed value of a parameter of the electrical stimulation has a value that is less than a predetermined maximum implantation-procedure value of the parameter, increasing the value of the parameter, and
if the programmed value of the parameter equals or exceeds the predetermined maximum implantation-procedure value of the parameter, adjusting the implant location of the nerve stimulator implant.

For some applications, the adverse-sensation site is skin in a vicinity of the implant location.

For some applications, the adverse sensation includes one or more sensations selected from the group consisting of: paresthesia and pain.

For some applications, the target nerve is a tibial nerve, and activating the nerve stimulator implant includes activating the nerve stimulator implant to apply the electrical stimulation to the portion of the tibial nerve.

For some applications, the remote-paresthesia site is on a plantar surface of a foot.

For some applications, the remote-paresthesia site includes one or more dermatomes selected from the group of dermatomes consisting of: a dermatome innervated by a medial calcaneal nerve, a dermatome innervated by a medial plantar nerve, and a dermatome innervated by a lateral plantar nerve.

For some applications, the adverse-sensation site is an Achilles tendon.

For some applications, the remote-paresthesia site is remote from a bladder of the patient.

For some applications, the remote-paresthesia site is more than 5 cm from the implant location.

For some applications, placing the nerve stimulator implant at the implant location includes surgically exposing the implant location.

For some applications, placing the nerve stimulator implant at the implant location includes injecting the nerve stimulator implant into the body.

There is further provided, in accordance with an application of the present invention, a method for performing configuration of an implanted nerve stimulator implant, the method including:
(i) activating the implanted nerve stimulator implant to apply electrical stimulation to a tibial nerve of a patient using a set of one or more stimulation parameters;
(ii) receiving, from the patient, feedback indicative of where on a plantar surface of a foot the patient feels paresthesia caused by the electrical stimulation;
(iii) based on the feedback, ascertaining at least a presence or an absence of remote-site paresthesia caused by the electrical stimulation at a predetermined dermatome of the plantar surface of the foot, the dermatome selected from the group of dermatomes consisting of: a dermatome innervated by a medial calcaneal nerve, a dermatome innervated by a medial plantar nerve, and a dermatome innervated by a lateral plantar nerve; and
(iv) based on the presence or the absence of the remote-site paresthesia at the predetermined dermatome, either:
   adjusting at least one of the one or more stimulation parameters and thereafter repeating steps (ii), (iii), and (iv) one or more times, or
   completing the configuration of the nerve stimulator implant without repeating steps (ii), (iii), or (iv).

For some applications, step (iv) includes:
based on the absence of the remote-site paresthesia at the predetermined dermatome, adjusting the at least one of the one or more stimulation parameters and thereafter repeating steps (ii), (iii), and (iv) the one or more times, or
based on the presence of the remote-site paresthesia at the predetermined dermatome, completing the configuration of the nerve stimulator implant without repeating steps (ii), (iii), or (iv).

For some applications:
the predetermined dermatome is a first predetermined dermatome,
ascertaining at step (iii) includes, based on the feedback, ascertaining:
   (a) at least the presence or the absence of the remote-site paresthesia caused by the electrical stimulation at the first predetermined dermatome of the plantar surface of the foot, and
   (b) at least a presence or an absence of remote-site paresthesia caused by the electrical stimulation at a second predetermined dermatome of the plantar surface of the foot, the second predetermined dermatome (1) different from the first predetermined dermatome, and (2) selected from the group of dermatomes consisting of: the dermatome innervated by the medial calcaneal nerve, the dermatome innervated by the medial plantar nerve, and the dermatome innervated by the lateral plantar nerve, and
step (iv) includes, based on the presence of the absence of the remote-site paresthesia at at least one of the first and the second predetermined dermatomes, either:
   adjusting at least one of the one or more stimulation parameters and thereafter repeating steps (ii), (iii), and (iv) one or more times, or
   completing the configuration of the nerve stimulator implant without repeating steps (ii), (iii), or (iv).

For some applications, step (iv) includes:
based on the absence of the remote-site paresthesia at at least one of the first and the second predetermined dermatomes, adjusting the at least one of the one or more stimulation parameters and thereafter repeating steps (ii), (iii), and (iv) the one or more times, or
based on the presence of the remote-site paresthesia at both the first and the second predetermined dermatomes, completing the configuration of the nerve stimulator implant without repeating steps (ii), (iii), or (iv).

For some applications:
ascertaining at step (iii) includes, based on the feedback, ascertaining:
   (a) at least the presence or the absence of the remote-site paresthesia caused by the electrical stimulation at the first predetermined dermatome of the plantar surface of the foot,
   (a) at least the presence or the absence of the remote-site paresthesia caused by the electrical stimulation at the second predetermined dermatome of the plantar surface of the foot, and
   (b) at least a presence or an absence of remote-site paresthesia caused by the electrical stimulation at a third predetermined dermatome of the plantar surface of the foot, the third predetermined dermatome (1) different from the first and the second predetermined dermatomes, and (2) selected from the group of dermatomes consisting of: the dermatome innervated by the medial calcaneal nerve, the dermatome innervated by the medial plantar nerve, and the dermatome innervated by the lateral plantar nerve, and
step (iv) includes, based on the presence or the absence of the remote-site paresthesia at at least one of the first, the second, and the third predetermined dermatomes, either:
   adjusting at least one of the one or more stimulation parameters and thereafter repeating steps (ii), (iii), and (iv) one or more times, or
   completing the configuration of the nerve stimulator implant without repeating steps (ii), (iii), or (iv).

For some applications, step (iv) includes:
based on the absence of the remote-site paresthesia at at least one of the first, the second, and the third predetermined dermatomes, adjusting the at least one of the one or more stimulation parameters and thereafter repeating steps (ii), (iii), and (iv) the one or more times, or
based on the presence of the remote-site paresthesia at all of the first, the second, and the third predetermined dermatomes, completing the configuration of the nerve stimulator implant without repeating steps (ii), (iii), or (iv).

For some applications, step (iv) includes:
based on the absence of the remote-site paresthesia at at least two of the first, the second, and the third predetermined dermatomes, adjusting the at least one of the one or more stimulation parameters and thereafter repeating steps (ii), (iii), and (iv) the one or more times, or
based on the presence of the remote-site paresthesia at all of the first, the second, and the third predetermined dermatomes, completing the configuration of the nerve stimulator implant without repeating steps (ii), (iii), or (iv).

For some applications, the one or more stimulation parameters include one or more parameters selected from the group consisting of: pulse width, frequency, amplitude, and polarity.

The present invention will be more fully understood from the following detailed description of embodiments thereof, taken together with the drawings, in which:

### BRIEF DESCRIPTION OF THE DRAWINGS

FIG. 1 is a flowchart schematically illustrating a method for performing an implantation of a nerve stimulator implant in a body of a patient, in accordance with an application of the present invention;

FIGS. 2A-D are schematic illustrations of the steps of one implementation of the method of FIG. 1, in accordance with an application of the present invention;

FIG. 3 is a flowchart schematically illustrating a method for performing configuration of an implanted nerve stimulator implant, in accordance with an application of the present invention; and

FIG. 4 is a schematic illustration of dermatomes of a plantar surface of a foot, as is known in anatomy.

### DETAILED DESCRIPTION OF APPLICATIONS

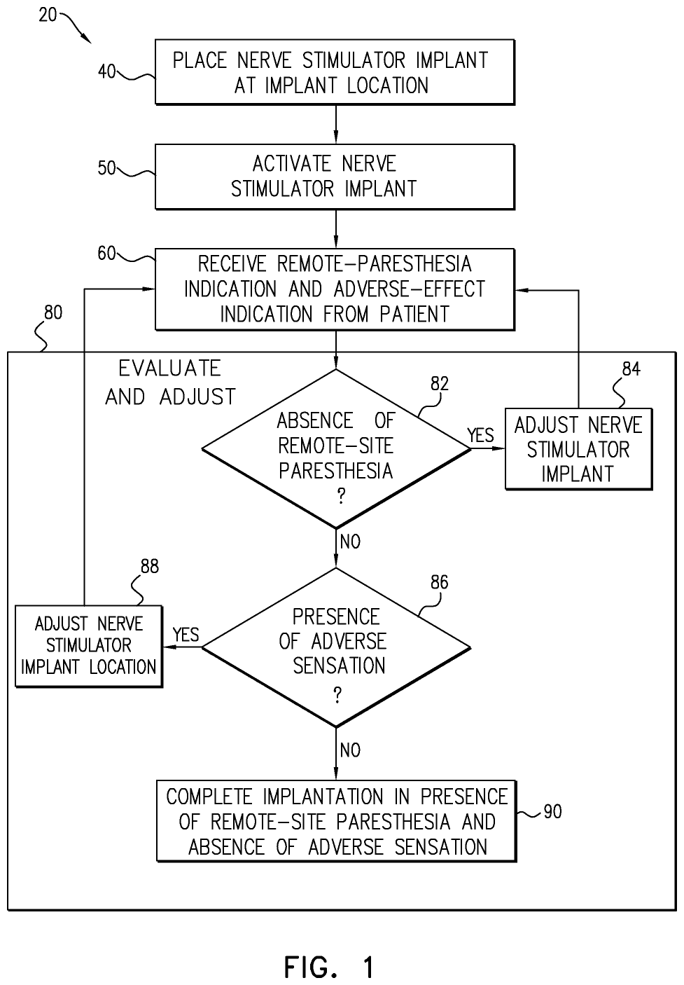

Reference is made to FIG. 1, which is a flowchart schematically illustrating a method 20 for performing an implantation of a nerve stimulator implant 30 in a body 32 of a patient, in accordance with an application of the present invention. Nerve stimulator implant 30 is illustrated by way of example and not limitation, and may comprise any implantable nerve stimulator implant. Optionally, nerve stimulator implant 30 implements techniques described in one or more of the patents and/or patent applications incorporated by reference hereinbelow.

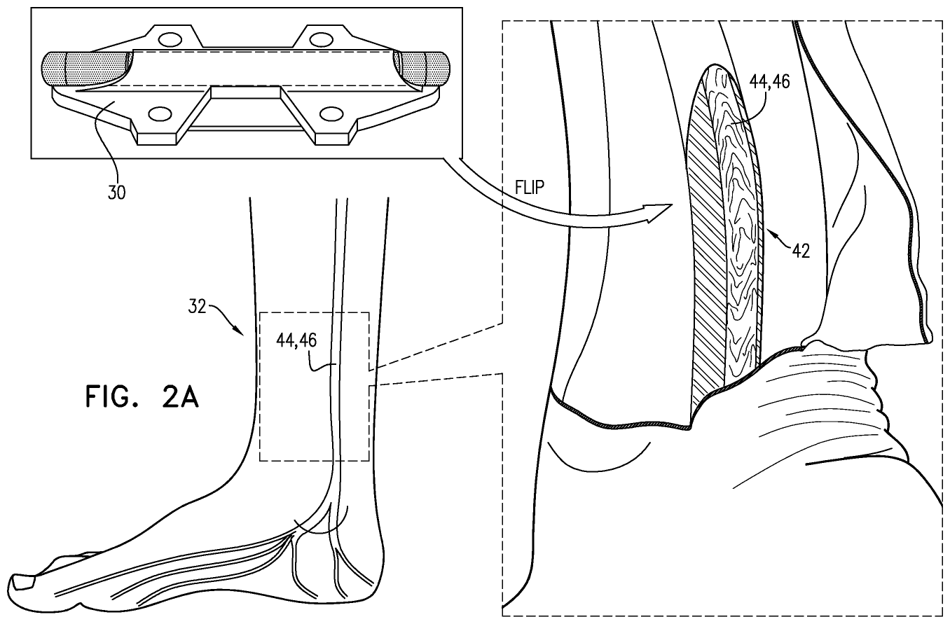

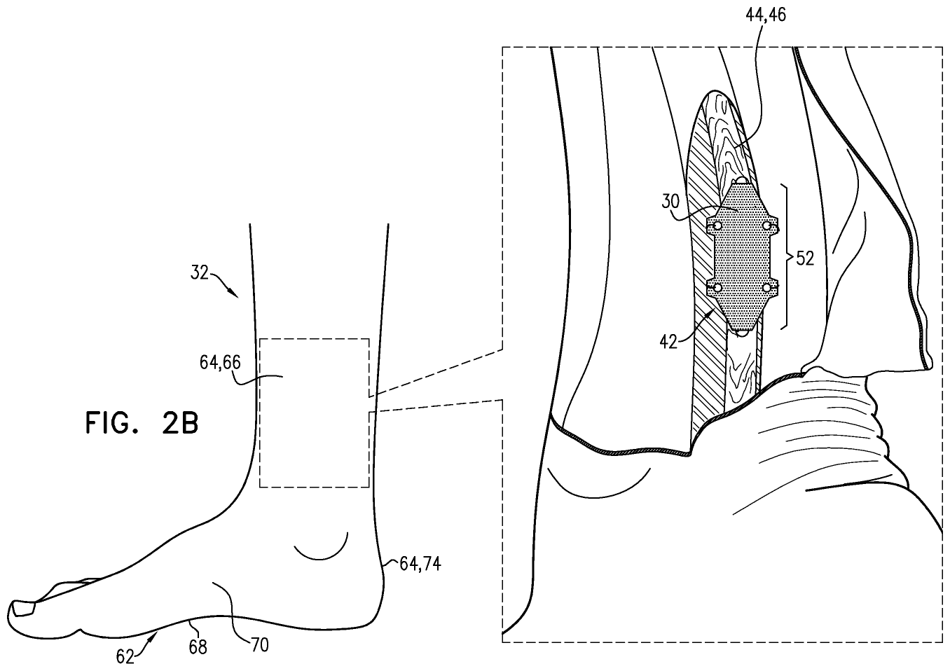

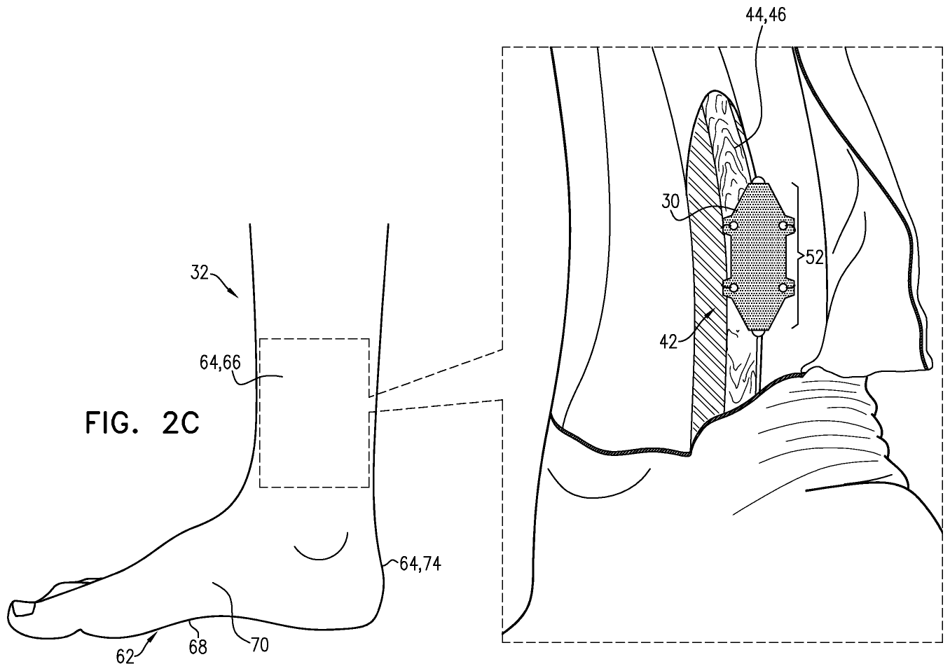

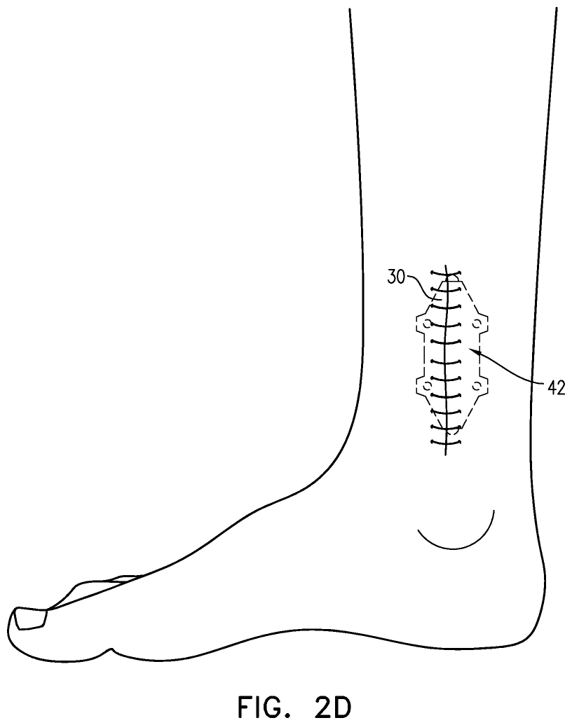

Reference is also made to FIGS. 2A-D, which are schematic illustrations of one implementation of the steps of method 20, in accordance with an application of the present invention.

Method 20 begins at a nerve stimulator implant placement step 40, at which a healthcare professional places nerve stimulator implant 30 at an implant location 42 in body 32 in a vicinity of a target nerve 44, such as a tibial nerve 46, such as shown in FIG. 2A. Typically, the healthcare professional is a physician, such as a surgeon.

For some applications, placing nerve stimulator implant 30 at implant location 42 comprises surgically exposing implant location 42, such as shown by way of example and not limitation in FIGS. 2A-C. For other applications, placing nerve stimulator implant 30 at implant location 42 comprises injecting nerve stimulator implant 30 into body 32, typically using a needle; for example, techniques may be used that are described in U.S. Pat. No. 9,861,812 to Gross et al., U.S. Pat. No. 9,713,707 to Oron et al., U.S. Pat. No. 10,004,896 to Oron et al., and/or U.S. Pat. No. 10,124,178 to Oron et al., all of which are incorporated herein by reference.

At an activation step 50, nerve stimulator implant 30 is activated to apply electrical stimulation to a portion 52 of target nerve 44, such as shown in FIG. 2B.

At a patient feedback step 60, the healthcare professional receives at least the following feedback from the patient:
a remote-paresthesia indication of at least a presence or an absence of remote-site paresthesia caused by the electrical stimulation at a predetermined remote-paresthesia site 62 of body 32 that is (1) innervated by portion 52 of target nerve 44, (2) remote from implant location 42, and (3) not a therapeutic target of stimulation by nerve stimulator implant 30, and
an adverse-effect indication of at least a presence or an absence of an adverse sensation caused by the electrical stimulation at an adverse-sensation site 64 of body 32 that is not innervated by portion 52 of target nerve 44.

Typically, remote-paresthesia site 62 is remote from a bladder of the patient.

In some patients, remote-paresthesia site 62 is more than 5 cm from implant location 42.

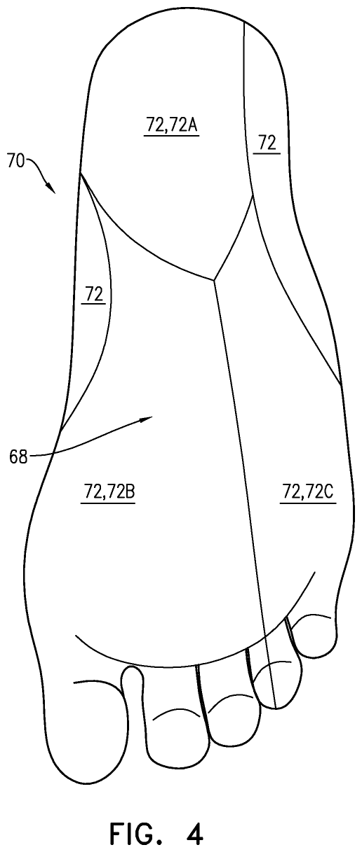

For some applications in which target nerve 44 is tibial nerve 46, remote-paresthesia site 62 is on a plantar surface 68 of a foot 70, in which case remote-paresthesia site 62 may include one or more dermatomes 72 of plantar surface 68 selected from the group of dermatomes 72 consisting of: a dermatome 72A innervated by a medial calcaneal nerve, a dermatome 72B innervated by a medial plantar nerve, and a dermatome 72C innervated by a lateral plantar nerve, as shown in FIG. 4, described hereinbelow. (Portion 52 of tibial nerve 46 to which nerve stimulator implant 30 applies the electrical stimulation innervates dermatomes 72A, 72B, and 72C.) As used in the present application, including in the claims, plantar surface 68 of foot 70 includes both the sole and plantar surfaces of toes of foot 70.

Although the electrical stimulation is generally described herein as causing remote-paresthesia, optionally the electrical stimulation alternatively or additionally causes other remote-site sensations, which may be used in the methods described herein in addition to, or instead of, paresthesia.

In general, adverse sensations occur when the electrical stimulation applied by nerve stimulator implant 30 undesirably stimulates nerves other than target nerve 44, or undesirably directly stimulates a muscle. In some patients, adverse-sensation site 64 is skin 66 in a vicinity of implant location 42, and the adverse sensation includes paresthesia and/or pain. Alternatively or additionally, adverse-sensation site 64 is remote from implant location 42, such as at least 1 cm from implant location 42. For example, for some applications in which target nerve 44 is tibial nerve 46, adverse-sensation site 64 is an Achilles tendon 74, which is generally indicative of undesirable muscle stimulation (patients sometimes refer to the Achilles tendon as their ankle). (It is noted that Achilles tendon 74 is innervated by a different, more afferent portion of tibial nerve 46 afferent to a branch that is afferent to portion 52 of tibial nerve 46 to which nerve stimulator implant 30 applies the electrical stimulation.)

Reference is still made to FIG. 1. At an evaluation and adjustment step 80:
at a remote-paresthesia check step 82, the healthcare professional evaluates the remote-paresthesia indication; in response to the remote-paresthesia indication being of the absence of the remote-site paresthesia, the healthcare professional adjusts nerve stimulator implant 30 at a nerve stimulator implant adjustment step 84, and thereafter repeats patient feedback step 60 and evaluation and adjustment step 80, and, optionally, activation step 50 if nerve stimulator implant 30 has been deactivated;
at an adverse-effect check step 86, the healthcare professional evaluates the adverse-effect indication; in response to the adverse-effect indication being of the presence of the adverse sensation, the healthcare professional adjusts implant location 42 of nerve stimulator implant 30 at an implant location adjustment step 88, and thereafter repeats patient feedback step 60 and evaluation and adjustment step 80, and, optionally, activation step 50 if nerve stimulator implant 30 has been deactivated;
in response to both (1) the remote-paresthesia indication being indicative of the presence of the remote-site paresthesia and (2) the adverse-effect indication being indicative of the absence of the adverse sensation, the healthcare professional completes the implantation of nerve stimulator implant 30 at implant location 42 at an implantation completion step 90, such as shown in FIG. 2D, without repeating patient feedback step 60 and evaluation and adjustment step 80.

The healthcare professional does not necessarily perform remote-paresthesia check step 82 and adverse-effect check step 86 at any given iteration of evaluation and adjustment step 80. For example, in response to the remote-paresthesia indication being of the absence of the remote-site paresthesia at remote-paresthesia check step 82, the healthcare professional does not necessarily perform adverse-effect check step 86 during the same iteration of evaluation and adjustment step 80.

For some applications, adjusting nerve stimulator implant 30 at nerve stimulator implant adjustment step 84 comprises adjusting implant location 42 of nerve stimulator implant 30, such as shown in FIG. 2C. For example, in applications in which nerve stimulator implant 30 is placed surgically, adjusting implant location 42 of nerve stimulator implant 30 may comprise moving implant 30 anteriorly or posteriorly (i.e., to the right or to the left). In applications in which nerve stimulator is injected, adjusting implant location 42 of nerve stimulator implant 30 may comprise moving implant 30 to a deeper or shallower implant location 42.

Alternatively or additionally, for some applications, adjusting nerve stimulator implant 30 at nerve stimulator implant adjustment step 84 comprises configuring one or more parameters of the electrical stimulation to increase an implantation-procedure strength the electrical stimulation applied by nerve stimulator implant 30. Typically, the one or more stimulation parameters include one or more parameters selected from the group consisting of: pulse width, frequency, amplitude, and polarity. In some configurations, the one or more parameters adjusted during the implantation procedure include only a single parameter, such as amplitude, for example; other stimulation parameters may be held constant throughout the implantation procedure.

For some applications, adjusting nerve stimulator implant 30 at nerve stimulator implant adjustment step 84 comprises:
if nerve stimulator implant 30 is programmed with one or more parameters of the electrical stimulation that produce a strength of the electrical stimulation that is less than a predetermined maximum implantation-procedure strength of the electrical stimulation, configuring the one or more parameters to increase the implantation-procedure strength of the electrical stimulation, and
if nerve stimulator implant 30 is programmed with the one or more parameters that produce the predetermined maximum implantation-procedure strength of the electrical stimulation, adjusting implant location 42 of nerve stimulator implant 30, such as shown in FIG. 2C.

Alternatively or additionally, for some applications, adjusting nerve stimulator implant 30 at nerve stimulator implant adjustment step 84 comprises:
if a programmed value of a parameter of the electrical stimulation has a value that is less than a predetermined maximum implantation-procedure value of the parameter, increasing the value of the parameter, and
if the programmed value of the parameter equals or exceeds the predetermined maximum implantation-procedure value of the parameter, adjusting implant location 42 of nerve stimulator implant 30, such as shown in FIG. 2C.

For some applications, the predetermined maximum implantation-procedure value of the parameter may be equal to a predetermined percentage of a predetermined maximum post-implantation value of the parameter, either for all patients or the particular patient. For example, the predetermined percentage may be 25%-75%, such as 50%.

In some applications in which placing nerve stimulator implant 30 at implant location 42 comprises surgically exposing implant location 42, completing the implantation of nerve stimulator implant 30 at implant location 42 at implantation completion step 90 comprises closing the surgical wound, such as shown in FIG. 2D. In other applications in which placing nerve stimulator implant 30 at implant location 42 comprises injecting nerve stimulator implant 30 into body 32, completing the implantation of nerve stimulator implant 30 at implant location 42 at implantation completion step 90 comprises removing the needle used for injection.

A clinical study was conducted using some of the techniques described herein with reference to FIGS. 1 and 2A-D. In about 30% of the 151 patients in which nerve stimulator implant 30 was implanted, the implant was relocated during the implantation procedure based on the feedback described herein. These experimental results demonstrate the usefulness of the techniques described herein with reference to FIGS. 1 and 2A-D for improving the clinical outcome of the implantation procedure.

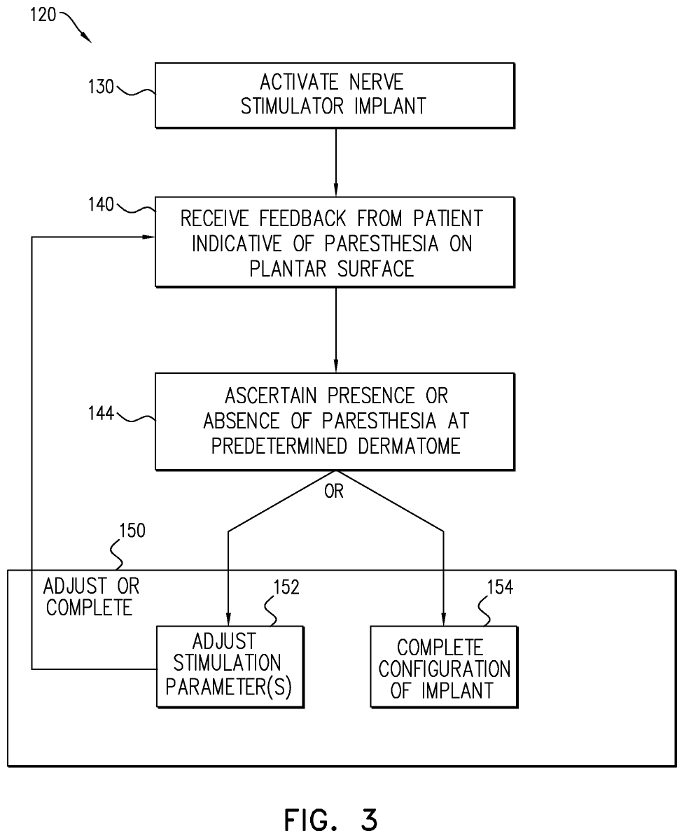

Reference is now made to FIG. 3, which is a flowchart schematically illustrating a method 120 for performing configuration of an implanted nerve stimulator implant, in accordance with an application of the present invention. The nerve stimulator implant may comprise nerve stimulator implant 30, or any other implantable nerve stimulator implant. Optionally, the nerve stimulator implant implements techniques described in one or more of the patents and/or patent applications incorporated by reference hereinbelow. The configuration may be performed, for example, during initial programming and/or reprogramming of the implanted nerve stimulator implant.

Reference is also made to FIG. 4, which is a schematic illustration of dermatomes 72 of plantar surface 68 of foot 70, as is known in anatomy.

Method 120 is typically performed after the nerve stimulator implant has been fully implanted, typically at least 28 days after completion of the implantation. For some applications, method 120 is performed after completion of method 20, described hereinabove with reference to FIGS. 1 and 2A-D, while for other applications, method 120 is performed independently of method 20. Although method 120 is generally described as being performed to configured an implanted nerve stimulator implant, the method may alternatively be used to configured a transcutaneous nerve stimulator, such as a TENS device.

Method 120 begins at an activation step 130, at which implanted the nerve stimulator implant is activated to apply electrical stimulation to tibial nerve 46 using a set of one or more stimulation parameters. The one or more stimulation parameters may be default stimulation parameters, or may be initial stimulation parameters set for the particular patient, such as during or after the implantation of the nerve stimulator implant.

In some configurations, the nerve stimulator implant is programmable with a plurality of treatment programs (e.g., 2-6 treatment programs), having respective sets of one or more stimulation parameters. The patient and/or healthcare professional can choose which of the treatment programs to activate at any given time. In some of these configurations, method 120 is separately performed for configurating at least one of the treatment programs, such as for each of the treatment programs.

In general, in performing method 120, the healthcare professional uses patient-reported paresthesia at one, two, or three predetermined dermatomes 72 of plantar surface 68 of foot 70 to adjust one or more stimulation parameters, in order to maximize coverage of the dermatomes 72. In some applications, this parameter adjustment includes a preference for a particular predetermined dermatome or two particular predetermined dermatomes if the parameter adjustment is not able to achieve paresthesia at all three predetermined dermatomes 72 after a reasonable number of attempts.

At a patient feedback step 140, the healthcare professional receives, from the patient, feedback indicative of where on plantar surface 68 of foot 70 the patient feels paresthesia caused by the electrical stimulation.

At a dermatome ascertaining step 144, based on the feedback, the healthcare professional ascertains at least a presence or an absence of remote-site paresthesia caused by the electrical stimulation at a predetermined dermatome 72 of plantar surface 68 of foot 70. Typically, the predetermined dermatome 72 is selected from the group of dermatomes 72 consisting of: dermatome 72A innervated by the medial calcaneal nerve, dermatome 72B innervated by the medial plantar nerve, and dermatome 72C innervated by the lateral plantar nerve, as shown in FIG. 4.

At an adjustment or completion step 150, based on the presence or the absence of the remote-site paresthesia at predetermined dermatome 72, either:
at an adjustment step 152, the healthcare professional adjusts at least one of the one or more stimulation parameters and thereafter repeats patient feedback step 140, dermatome ascertaining step 144, and adjustment or completion step 150 one or more times, and, optionally, activation step 130 if the nerve stimulator implant has been deactivated, or
at a completion step 154, the healthcare professional adjusts completes the configuration of the nerve stimulator implant without repeating patient feedback step 140, dermatome ascertaining step 144, or adjustment or completion step 150.

Typically, the one or more stimulation parameters include one or more parameters selected from the group consisting of: pulse width, frequency, amplitude, and polarity.

For some applications, at adjustment or completion step 150:
at adjustment step 152, based on the absence of the remote-site paresthesia at predetermined dermatome 72, the healthcare professional adjusts the at least one of the one or more stimulation parameters and thereafter repeats patient feedback step 140, dermatome ascertaining step 144, and adjustment or completion step 150 the one or more times, and, optionally, activation step 130 if the nerve stimulator implant has been deactivated, or
at completion step 154, based on the presence of the remote-site paresthesia at predetermined dermatome 72, the healthcare professional completes the configuration of the nerve stimulator implant without repeating patient feedback step 140, dermatome ascertaining step 144, or adjustment or completion step 150.

In these applications, at adjustment step 152, based on the absence of the remote-site paresthesia at predetermined dermatome 72, the healthcare professional adjusts the at least one of the one or more stimulation parameters so as to increase a strength of the stimulation applied by the nerve stimulator implant. (Optionally, during the implantation of the nerve stimulator implant prior to performance of method 120, the strength of the stimulation is set at a fraction of the maximum strength, e.g., 30%-80%, such as 50%, of the maximum strength, in order to provide flexibility in adjusting the stimulation parameters post-implantation, such as using method 120.)

For some applications, predetermined dermatome 72 is a first predetermined dermatome 72-1, and at dermatome ascertaining step 144, based on the feedback, the healthcare professional ascertains:
at least the presence or the absence of the remote-site paresthesia caused by the electrical stimulation at first predetermined dermatome 72-1 of plantar surface 68 of foot 70, and
at least a presence or an absence of remote-site paresthesia caused by the electrical stimulation at a second predetermined dermatome 72-2 of plantar surface 68 of foot 70, second predetermined dermatome 72-2 (a) different from first predetermined dermatome 72-1, and (b) selected from the group of dermatomes consisting of: dermatome 72A innervated by the medial calcaneal nerve, dermatome 72B innervated by the medial plantar nerve, and dermatome 72C innervated by the lateral plantar nerve, as shown in FIG. 4.

For these applications, at adjustment or completion step 150, based on the presence or the absence of the remote-site paresthesia at at least one of first predetermined dermatome 72-1 and second predetermined dermatome 72-2, either:
at adjustment step 152, the healthcare professional adjusts at least one of the one or more stimulation parameters and thereafter repeats patient feedback step 140, dermatome ascertaining step 144, and adjustment or completion step 150 the one or more times, and, optionally, activation step 130 if the nerve stimulator implant has been deactivated, or
at completion step 154, the healthcare professional completes the configuration of the nerve stimulator implant without repeating patient feedback step 140, dermatome ascertaining step 144, or adjustment or completion step 150.

For some of these applications, adjustment or completion step 150 comprises:
at adjustment step 152, based on the absence of the remote-site paresthesia at at least one of first predetermined dermatome 72-1 and second predetermined dermatome 72-2 (optionally, based on the absence of the remote-site paresthesia at both of first predetermined dermatome 72-1 and second predetermined dermatome 72-2), adjusting the at least one of the one or more stimulation parameters and thereafter repeating patient feedback step 140, dermatome ascertaining step 144, and adjustment or completion step 150 the one or more times, and, optionally, activation step 130 if the nerve stimulator implant has been deactivated, or
based on the presence of the remote-site paresthesia at both first predetermined dermatome 72-1 and second predetermined dermatome 72-2, completing the configuration of the nerve stimulator implant without repeating patient feedback step 140, dermatome ascertaining step 144, or adjustment or completion step 150.

Alternatively, the configuration of the nerve stimulator implant is completed based on the presence of only one of first predetermined dermatome 72-1 and second predetermined dermatome 72-2.

For some applications, at dermatome ascertaining step 144, based on the feedback, the healthcare professional ascertains:
at least the presence or the absence of the remote-site paresthesia caused by the electrical stimulation at first predetermined dermatome 72-1 of plantar surface 68 of foot 70,
at least the presence or the absence of the remote-site paresthesia caused by the electrical stimulation at second predetermined dermatome 72-2 of plantar surface 68 of foot 70, and
at least a presence or an absence of remote-site paresthesia caused by the electrical stimulation at a third predetermined dermatome 72-3 of plantar surface 68 of foot 70, third predetermined dermatome 72-3 (a) different from first predetermined dermatome 72-1 and second predetermined dermatome 72-2, and (b) selected from the group of dermatomes consisting of: dermatome 72A innervated by the medial calcaneal nerve, dermatome 72B innervated by the medial plantar nerve, and dermatome 72C innervated by the lateral plantar nerve, and

For these applications, at adjustment or completion step 150, based on the presence or the absence of the remote-site paresthesia at at least one of first predetermined dermatome 72-1, second predetermined dermatome 72-1, and third predetermined dermatome 72-3, either:
at adjustment step 152, the healthcare professional adjusts at least one of the one or more stimulation parameters and thereafter repeats patient feedback step 140, dermatome ascertaining step 144, and adjustment or completion step 150 the one or more times, and, optionally, activation step 130 if the nerve stimulator implant has been deactivated, or
at completion step 154, the healthcare professional completes the configuration of the nerve stimulator implant without repeating patient feedback step 140, dermatome ascertaining step 144, or adjustment or completion step 150.

For some of these applications, adjustment or completion step 150 comprises:
at adjustment step 152, based on the absence of the remote-site paresthesia at at least one of first predetermined dermatome 72-1, second predetermined dermatome 72-1, and third predetermined dermatome 72-3 (optionally, based on the absence of the remote-site paresthesia at (a) two of first predetermined dermatome 72-1, second predetermined dermatome 72-2, and third predetermined dermatome 72-3, or (b) all three of first predetermined dermatome 72-1, second predetermined dermatome 72-2, and third predetermined dermatome 72-3), adjusting the at least one of the one or more stimulation parameters and thereafter repeating patient feedback step 140, dermatome ascertaining step 144, and adjustment or completion step 150 the one or more times, and, optionally, activation step 130 if the nerve stimulator implant has been deactivated, or
based on the presence of the remote-site paresthesia at all of first predetermined dermatome 72-1, second predetermined dermatome 72-2, and third predetermined dermatome 72-3, completing the configuration of the nerve stimulator implant without repeating patient feedback step 140, dermatome ascertaining step 144, or adjustment or completion step 150.

Although several steps of the methods described herein have been described as being performed by a healthcare professional, some or all of these steps may be otherwise performed, such as by hardware and/or software, including, but not limited to, a robotic system.

In an embodiment, techniques and apparatus described in one or more of the following applications are combined with techniques and apparatus described herein:
U.S. Pat. No. 8,788,045 to Gross et al.
U.S. Pat. No. 8,755,893 to Gross et al.
PCT Publication WO 2013/111137 to Gross et al., and U.S. Patent Application Publication 2015/0018728 in the U.S. national stage thereof
PCT Publication WO 2014/087337 to Gross et al., and U.S. Pat. No. 9,861,812 in the U.S. national stage thereof
U.S. Pat. No. 9,597,521 to Plotkin et al.
U.S. Pat. No. 9,713,707 to Oron et al.
U.S. Pat. No. 9,764,146 to Oron et al.
U.S. Pat. No. 10,105,540 to Oron et al.
U.S. Pat. No. 10,124,178 to Oron et al.
U.S. Pat. No. 10,004,896 to Oron et al.
U.S. Pat. No. 11,213,685 to Oron et al.
U.S. patent application Ser. No. 17/838,655, filed Jun. 13, 2022, which published as US Patent Application Publication 2023/0122706 to Lemaitre et al.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method for performing an implantation of a nerve stimulator implant in a body of a patient, the method comprising:
(i) placing the nerve stimulator implant at an implant location in the body in a vicinity of a target nerve;
(ii) activating the nerve stimulator implant to apply electrical stimulation to a portion of the target nerve;
(iii) receiving from the patient:
(a) a remote-paresthesia indication of at least a presence or an absence of remote-site paresthesia caused by the electrical stimulation at a predetermined remote-paresthesia site of the body that is (1) innervated by the portion of the target nerve, (2) remote from the implant location, and (3) not a therapeutic target of stimulation by the nerve stimulator implant, and
(b) an adverse-effect indication of at least a presence or an absence of an adverse sensation caused by the electrical stimulation at an adverse-sensation site of the body that is not innervated by the portion of the target nerve; and
(iv) in response to:
(a) the remote-paresthesia indication being of the absence of the remote-site paresthesia, adjusting the nerve stimulator implant and thereafter repeating steps (iii) and (iv),
(b) the adverse-effect indication being of the presence of the adverse sensation, adjusting the implant location of the nerve stimulator implant and thereafter repeating steps (iii) and (iv), and
(c) both (1) the remote-paresthesia indication being indicative of the presence of the remote-site paresthesia and (2) the adverse-effect indication being indicative of the absence of the adverse sensation, completing the implantation of the nerve stimulator implant at the implant location without repeating steps (iii) or (iv).

2. The method according to claim 1, wherein adjusting the nerve stimulator implant at step (iv) (a) comprises adjusting the implant location of the nerve stimulator implant.

3. The method according to claim 1, wherein adjusting the nerve stimulator implant at step (iv) (a) comprises configuring one or more parameters of the electrical stimulation to increase an implantation-procedure strength the electrical stimulation applied by the nerve stimulator implant.

4. The method according to claim 1, wherein adjusting the nerve stimulator implant at step (iv) (a) comprises:
if the nerve stimulator implant is programmed with one or more parameters of the electrical stimulation that produce an implantation-procedure strength of the electrical stimulation that is less than a predetermined maximum implantation-procedure strength of the electrical stimulation, configuring the one or more parameters to increase the implantation-procedure strength the electrical stimulation, and
if the nerve stimulator implant is programmed with the one or more parameters that produce the predetermined maximum implantation-procedure strength of the electrical stimulation, adjusting the implant location of the nerve stimulator implant.

5. The method according to claim 1, wherein adjusting the nerve stimulator implant at step (iv) (a) comprises:
if a programmed value of a parameter of the electrical stimulation has a value that is less than a predetermined maximum implantation-procedure value of the parameter, increasing the value of the parameter, and
if the programmed value of the parameter equals or exceeds the predetermined maximum implantation-procedure value of the parameter, adjusting the implant location of the nerve stimulator implant.

6. The method according to claim 1, wherein the adverse-sensation site is skin in a vicinity of the implant location.

7. The method according to claim 6, wherein the adverse sensation includes one or more sensations selected from the group consisting of: paresthesia and pain.

8. The method according to claim 1, wherein the target nerve is a tibial nerve, and wherein activating the nerve stimulator implant comprises activating the nerve stimulator implant to apply the electrical stimulation to the portion of the tibial nerve.

9. The method according to claim 8, wherein the remote-paresthesia site is on a plantar surface of a foot.

10. The method according to claim 9, wherein the remote-paresthesia site includes one or more dermatomes selected from the group of dermatomes consisting of: a dermatome innervated by a medial calcaneal nerve, a dermatome innervated by a medial plantar nerve, and a dermatome innervated by a lateral plantar nerve.

11. The method according to claim 8, wherein the adverse-sensation site is an Achilles tendon.

12. The method according to claim 1, wherein the remote-paresthesia site is remote from a bladder of the patient.

13. The method according to claim 1, wherein the remote-paresthesia site is more than 5 cm from the implant location.

14. The method according to claim 1, wherein placing the nerve stimulator implant at the implant location comprises surgically exposing the implant location.

15. The method according to claim 1, wherein placing the nerve stimulator implant at the implant location comprises injecting the nerve stimulator implant into the body.

16. A method for performing configuration of an implanted nerve stimulator implant, the method comprising:
(i) activating the implanted nerve stimulator implant to apply electrical stimulation to a tibial nerve of a patient using a set 4 one or more stimulation parameters;
(ii) receiving, from the patient, feedback indicative of where on a plantar surface of a foot the patient feels paresthesia caused by the electrical stimulation;
(iii) based on the feedback, ascertaining at least a presence or an absence of remote-site paresthesia caused by the electrical stimulation at a predetermined dermatome of the plantar surface of the foot, the dermatome selected from the group of dermatomes consisting of: a dermatome innervated by a medial calcaneal nerve, a dermatome innervated by a medial plantar nerve, and a dermatome innervated by a lateral plantar nerve; and
(iv) based on the presence or the absence of the remote-site paresthesia at the predetermined dermatome, either:
adjusting at least one of the one or more stimulation parameters and thereafter repeating steps (ii), (iii), and (iv) one or more times, or
completing the configuration of the nerve stimulator implant without repeating steps (ii), (iii), or (iv).

17. The method according to claim 16, wherein step (iv) comprises:
based on the absence of the remote-site paresthesia at the predetermined dermatome, adjusting the at least one of the one or more stimulation parameters and thereafter repeating steps (ii), (iii), and (iv) the one or more times, or
based on the presence of the remote-site paresthesia at the predetermined dermatome, completing the configuration of the nerve stimulator implant without repeating steps (ii), (iii), or (iv).

18. The method according to claim 16,
wherein the predetermined dermatome is a first predetermined dermatome,
wherein ascertaining at step (iii) comprises, based on the feedback, ascertaining:
(a) at least the presence or the absence of the remote-site paresthesia caused by the electrical stimulation at the first predetermined dermatome of the plantar surface of the foot, and
(b) at least a presence or an absence of remote-site paresthesia caused by the electrical stimulation at a second predetermined dermatome of the plantar surface of the foot, the second predetermined dermatome (1) different from the first predetermined dermatome, and (2) selected from the group of dermatomes consisting of: the dermatome innervated by the medial calcaneal nerve, the dermatome innervated by the medial plantar nerve, and the dermatome innervated by the lateral plantar nerve, and
wherein step (iv) comprises, based on the presence or the absence of the remote-site paresthesia at at least one of the first and the second predetermined dermatomes, either:
adjusting at least one of the one or more stimulation parameters and thereafter repeating steps (ii), (iii), and (iv) one or more times, or
completing the configuration of the nerve stimulator implant without repeating steps (ii), (iii), or (iv).

19. The method according to claim 18, wherein step (iv) comprises:
based on the absence of the remote-site paresthesia at at least one of the first and the second predetermined dermatomes, adjusting the at least one of the one or more stimulation parameters and thereafter repeating steps (ii), (iii), and (iv) the one or more times, or
based on the presence of the remote-site paresthesia at both the first and the second predetermined dermatomes, completing the configuration of the nerve stimulator implant without repeating steps (ii), (iii), or (iv).

20. The method according to claim 18,
wherein ascertaining at step (iii) comprises, based on the feedback, ascertaining:
(a) at least the presence or the absence of the remote-site paresthesia caused by the electrical stimulation at the first predetermined dermatome of the plantar surface of the foot,
(a) at least the presence or the absence of the remote-site paresthesia caused by the electrical stimulation at the second predetermined dermatome of the plantar surface of the foot, and
(b) at least a presence or an absence of remote-site paresthesia caused by the electrical stimulation at a third predetermined dermatome of the plantar surface of the foot, the third predetermined dermatome (1) different from the first and the second predetermined dermatomes, and (2) selected from the group of dermatomes consisting of: the dermatome innervated by the medial calcaneal nerve, the dermatome innervated by the medial plantar nerve, and the dermatome innervated by the lateral plantar nerve, and
wherein step (iv) comprises, based on the presence or the absence of the remote-site paresthesia at at least one of the first, the second, and the third predetermined dermatomes, either:
adjusting at least one of the one or more stimulation parameters and thereafter repeating steps (ii), (iii), and (iv) one or more times, or
completing the configuration of the nerve stimulator implant without repeating steps (ii), (iii), or (iv).

21. The method according to claim 20, wherein step (iv) comprises:
based on the absence of the remote-site paresthesia at at least one of the first, the second, and the third predetermined dermatomes, adjusting the at least one of the one or more stimulation parameters and thereafter repeating steps (ii), (iii), and (iv) the one or more times, or
based on the presence of the remote-site paresthesia at all of the first, the second, and the third predetermined dermatomes, completing the configuration of the nerve stimulator implant without repeating steps (ii), (iii), or (iv).

22. The method according to claim 20, wherein step (iv) comprises:
based on the absence of the remote-site paresthesia at at least two of the first, the second, and the third predetermined dermatomes, adjusting the at least one of the one or more stimulation parameters and thereafter repeating steps (ii), (iii), and (iv) the one or more times, or
based on the presence of the remote-site paresthesia at all of the first, the second, and the third predetermined dermatomes, completing the configuration of the nerve stimulator implant without repeating steps (ii), (iii), or (iv).

23. The method according to claim 16, wherein the one or more stimulation parameters include one or more parameters selected from the group consisting of: pulse width, frequency, amplitude, and polarity.
